(19) United States
(12) Patent Application Publication
(10) Pub. No.: US 2017/0219545 A1
(43) Pub. Date: Aug. 3, 2017

(54) PRODUCE ITEM RIPENESS DETERMINATION

(71) Applicant: EMPIRE TECHNOLOGY DEVELOPMENT LLC, Wilmington, DE (US)

(72) Inventor: Masaaki MIHARA, Chiba (JP)

(21) Appl. No.: 15/013,917

(22) Filed: Feb. 2, 2016

(51) Int. Cl. G01N 33/02; G01N 27/22

(52) U.S. Cl. CPC G01N 33/025; G01N 27/228

(57) ABSTRACT

In some examples, a method is described. The method may include obtaining an electrical signal as an input signal. The method may also include generating a reference signal based on the input signal. Moreover, the method may include generating a device-under-test signal based on the input signal and based on a device-under-test capacitance between a first contact point and a second contact point. The first contact point may be electrically coupled to a first contact mechanism configured to contact a first contact area of the device-under-test. The second contact point may be configured to be electrically coupled to a second contact mechanism configured to contact a second contact area of the device-under-test. The method may also include generating a comparison signal based on a comparison between the reference signal and the device-under-test signal. Additionally, the method may include emitting an output signal that is based on the comparison signal.

### BACKGROUND

Unless otherwise indicated, the materials described in the present disclosure are not prior art to the claims in the present application and are not admitted to be prior art by inclusion in this section.

Produce items such as fruit are often sorted, shipped, sold, etc., based on ripeness of the produce items. However, many ripeness determination techniques cause damage to the produce items.

### SUMMARY

Technologies described in the present disclosure generally relate to produce item hardness measurements.

In some examples, a measurement circuit is described. The measurement circuit may include an input circuit configured to obtain an electrical signal as an input signal. The measurement circuit may also include a sensing circuit electrically coupled to the input circuit. The sensing circuit may be configured to generate a reference signal based on the input signal. The sensing circuit may also be configured to generate a device-under-test signal based on the input signal and based on a device-under-test capacitance between a first contact point of the sensing circuit and a second contact point of the sensing circuit. The first contact point may be electrically coupled to a first contact mechanism configured to contact a first contact area of a device-under-test. The second contact point may be configured to be electrically coupled to a second contact mechanism configured to contact a second contact area of the device-under-test. The measurement circuit may also include a comparison circuit electrically coupled to the sensing circuit and configured to generate a comparison signal based on a comparison between the reference signal and the device-under-test signal. In addition, the measurement circuit may include an output circuit electrically coupled to the comparison circuit and configured to emit an output signal that is based on the comparison signal.

In some examples, a measurement system is described. The measurement system may include a contact system. The contact system may include a first contact mechanism configured to contact a produce item at a first contact area of a surface of the produce item. The first contact mechanism may be electrically conductive and may be configured such that a size of the first contact area in contact with the first contact mechanism changes according to a ripeness of the produce item. The contact system may also include a second contact mechanism configured to contact the produce item at a second contact area of the surface of the produce item. The second contact mechanism may be electrically conductive. The measurement system may also include a measurement circuit that includes a first contact point electrically coupled to the first contact mechanism. The measurement circuit may also include a second contact point electrically coupled to the second contact mechanism. The measurement circuit may be configured to generate an output signal based on an electrical property between the first contact point and the second contact point.

In some examples, a method is described. The method may include obtaining an electrical signal as an input signal. The input signal may include an alternating current (AC) signal with one or more of the following based on a device-under-test: a peak-to-peak voltage and a frequency. The method may also include generating a reference signal based on the input signal. In addition, the method may include generating a device-under-test signal based on the input signal and based on a device-under-test capacitance between a first contact point and a second contact point. The first contact point may be electrically coupled to a first contact mechanism configured to contact a first contact area of the device-under-test. The second contact point may be configured to be electrically coupled to a second contact mechanism configured to contact a second contact area of the device-under-test. The method may also include generating a comparison signal based on a comparison between the reference signal and the device-under-test signal. In addition, the method may include emitting an output signal that is based on the comparison signal.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings. In the drawings:

FIG. 1 illustrates a block diagram of an example system configured to determine ripeness of a produce item;

FIG. 2 illustrates a block diagram of an example measurement circuit;

FIG. 3 illustrates an example measurement circuit;

FIG. 4 illustrates another example measurement circuit;

FIG. 5 illustrates another example measurement circuit;

FIG. 6A illustrates an example contact system;

FIG. 6B illustrates example gaps between a surface of a contact mechanism of the contact system of FIG. 6A and the surface of a produce item according to ripeness of the produce item;

FIG. 6C illustrates example sizes of contact areas of a contact mechanism of the contact system of FIG. 6A with the surface of the produce item according to ripeness of the produce item;

FIG. 7A illustrates another example contact system;

FIG. 7B illustrates example sizes of contact areas of a contact mechanism of the contact system of FIG. 7A with the surface of a produce item according to ripeness of the produce item;

FIG. 8 illustrates a flow diagram of an example method of determining a ripeness of a produce item; and

FIG. 9 is a block diagram illustrating an example computing system that is arranged to direct one or more operations associated with determining ripeness of a produce item;

all arranged in accordance with at least some embodiments described in the present disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, and devices that relate to determining ripeness of a produce item, which may be indicated by hardness of the produce item in some embodiments. As detailed below, the ripeness determination may be performed such that damage to the produce item may be reduced or negligible, as compared to other ripeness measurement techniques that may damage the produce item. In particular, as described below, systems and methods may be implemented in which one or more electrical properties associated with the produce items may be used to indicate ripeness of produce items.

Reference is now made to the drawings.

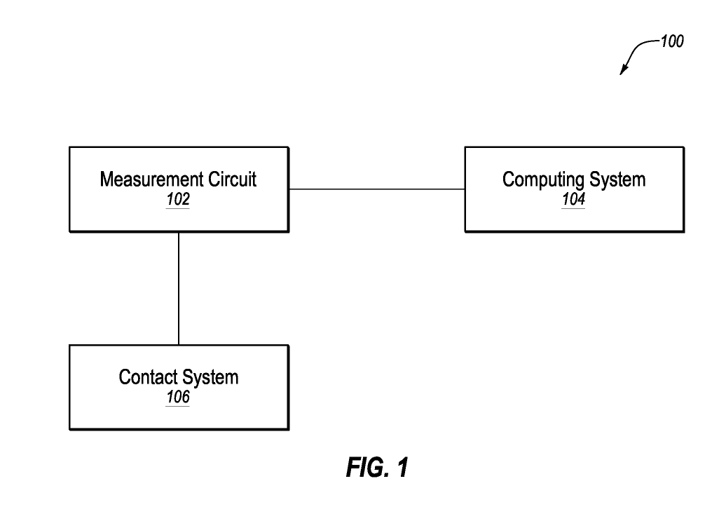

FIG. 1 illustrates a block diagram of an example system 100 configured to determine ripeness of a produce item, arranged in accordance with at least some embodiments described in the present disclosure. In the illustrated embodiment, the system 100 may include a measurement circuit 102, a computing system 104, and a contact system 106.

The contact system 106 may include any suitable system, apparatus, or device configured to contact a produce item in a manner such that an electrical property (e.g., impedance) measurement may be obtained with respect to the produce item. Further, the contact system 106 may be configured such that the electrical property measurement may indicate a ripeness of the produce item.

In some embodiments, the contact system 106 may include a first contact mechanism and a second contact mechanism configured to contact the produce item at first and second contact areas, respectively, of the produce item. Additionally or alternatively, the first contact mechanism may be configured such that a size of the first contact area in contact with the first contact mechanism changes according to the hardness of the produce item. In these or other embodiments, the second contact mechanism may be configured such that a size of the second contact area in contact with the second contact mechanism may also change according to the hardness of the produce item.

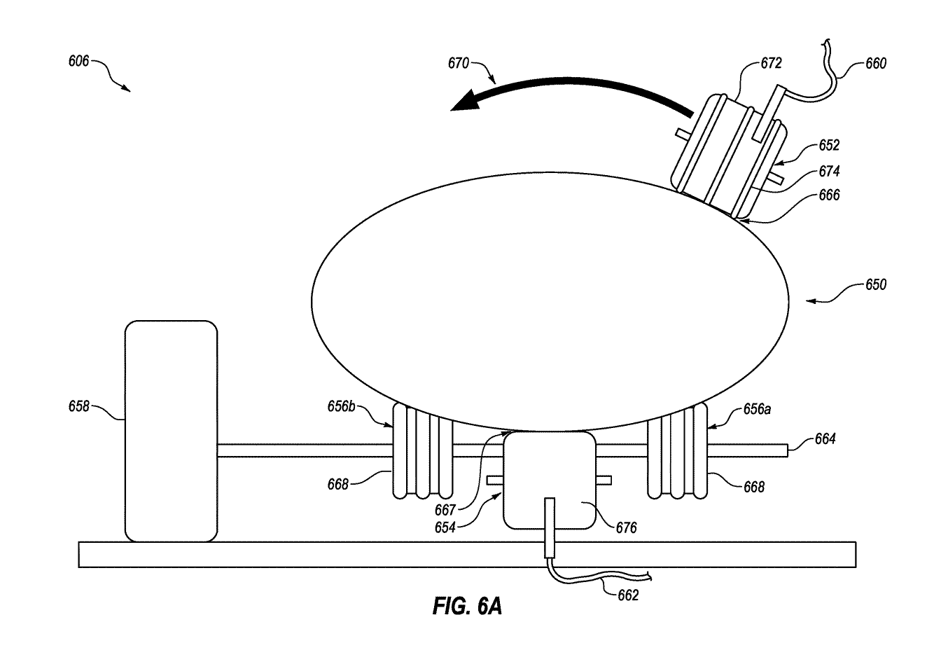

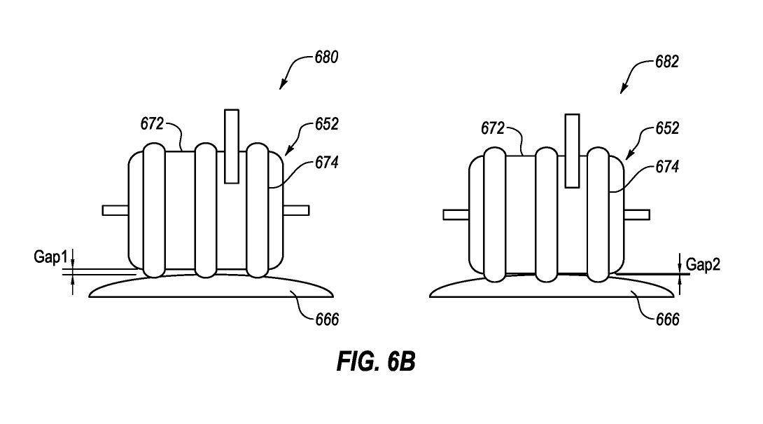

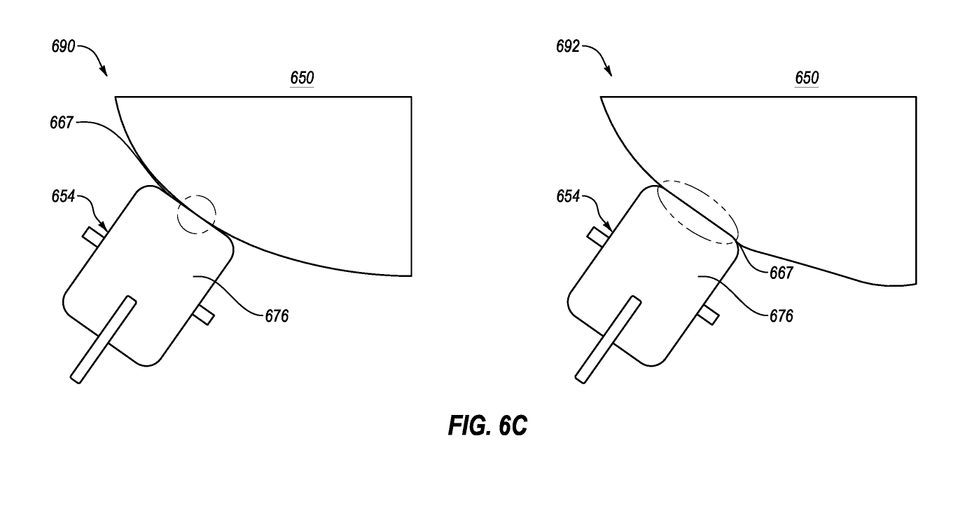

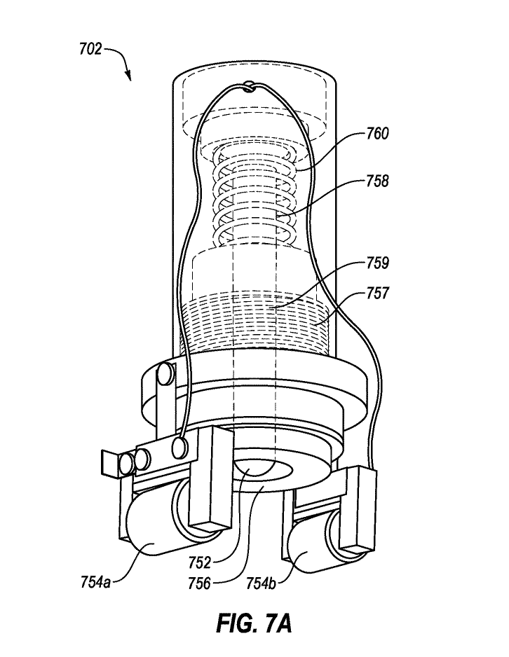

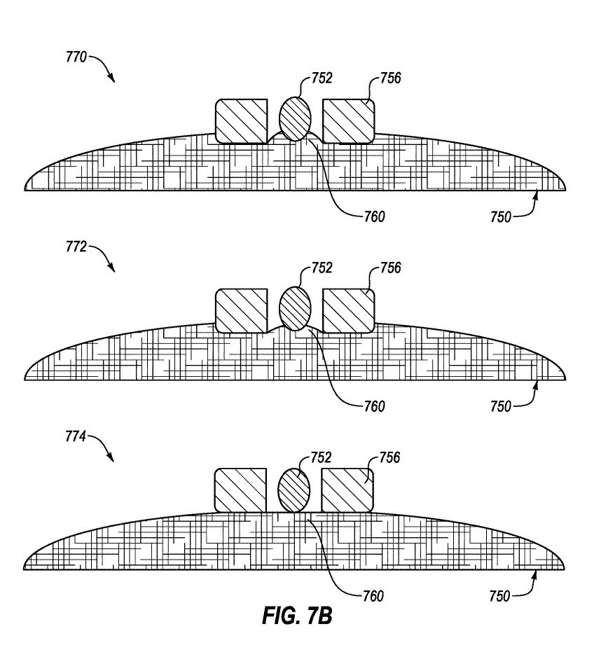

As described below, in some instances, the size of a contact area in contact with a corresponding contact mechanism may affect an electrical property (e.g., impedance, capacitance, etc.) of the produce item between the first and second contact mechanisms. Further, varying degrees of ripeness of the produce item may affect the electrical properties of the produce item between the first and second contact mechanisms in a manner that may affect the electrical property of the produce item between the first and second contact mechanisms. As such, the contact system 106 may be configured such that electrical property between the first and second contact mechanisms may be measured in a manner that indicates ripeness of the produce item. Examples of the contact system 106 are given below with respect to FIGS. 6A-6C and 7A-7B.

The measurement circuit 102 may include any suitable circuit configured to generate an output signal that may indicate an electrical property of a device-under-test such as a produce item. In some embodiments, the electrical property of the device-under-test (e.g., the produce item) may indicate a hardness or ripeness of the device-under-test such that the output signal may indicate the hardness or ripeness of the device-under-test.

For example, in some embodiments, the measurement circuit 102 may include a first contact point electrically coupled to the first contact mechanism of the contact system 106. Additionally, the measurement circuit 102 may include a second contact point electrically coupled to the second contact mechanism of the contact system 106. In some embodiments, the measurement circuit 102 may be configured to generate the output signal based on an electrical property between the first contact point and the second contact point. In these or other embodiments, the electrical property between the first contact point and the second contact point may be represented by a capacitance (which may indicate impedance) between the first contact point and the second contact point. Due to the electrical coupling of the first and second contact points with the first and second contact mechanisms, the electrical property between the first contact point and the second contact point may be based on the ripeness of the produce item.

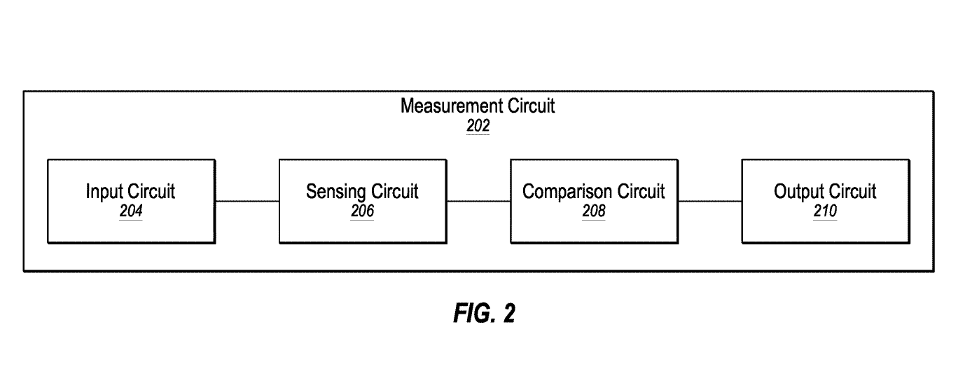

In particular, as mentioned above, the electrical properties of the produce item may change as the produce item ripens and gets softer such that the electrical property (e.g., capacitance, impedance, etc.) between the first contact point and the second contact point may change. Additionally or alternatively, as indicated above, the electrical property (e.g., between the first and second contact points may vary based on the size of a contact area in contact with a corresponding contact mechanism, which may be affected by the hardness and ripeness of the produce item. As such, the output signal may indicate the hardness of the produce item because of the dependence of the output signal on the electrical property between the first and second contact points. Examples of the measurement circuit 102 are given below with respect to FIGS. 2-5.

The computing system 104 may be communicatively coupled to the measurement circuit 102 such that it may receive the output signal generated by the measurement circuit 102. The computing system 104 may include any suitable system, apparatus, or device that may be configured to indicate a hardness of the produce item based on the output signal generated by the measurement circuit 102.

For example, in some embodiments, the computing system 104 may be configured to compare the output signal with a look-up table that correlates different values of the output signal with ripeness of the produce item. Based on the comparison, the computing system 104 may output an indication of ripeness (e.g., hardness) depending on the ripeness in the look-up table that correlates with an output signal in the look-up table that is closest to the output signal output by the measurement circuit 102.

In some embodiments, the look-up table may be generated by obtaining an output signal for different produce items that have different hardness or ripeness and then obtaining ripeness or hardness of the different produce items using other techniques (e.g., damaging techniques) used for making ripeness determinations. The output signal and corresponding hardness or ripeness may then be linked with each other and stored in the look-up table.

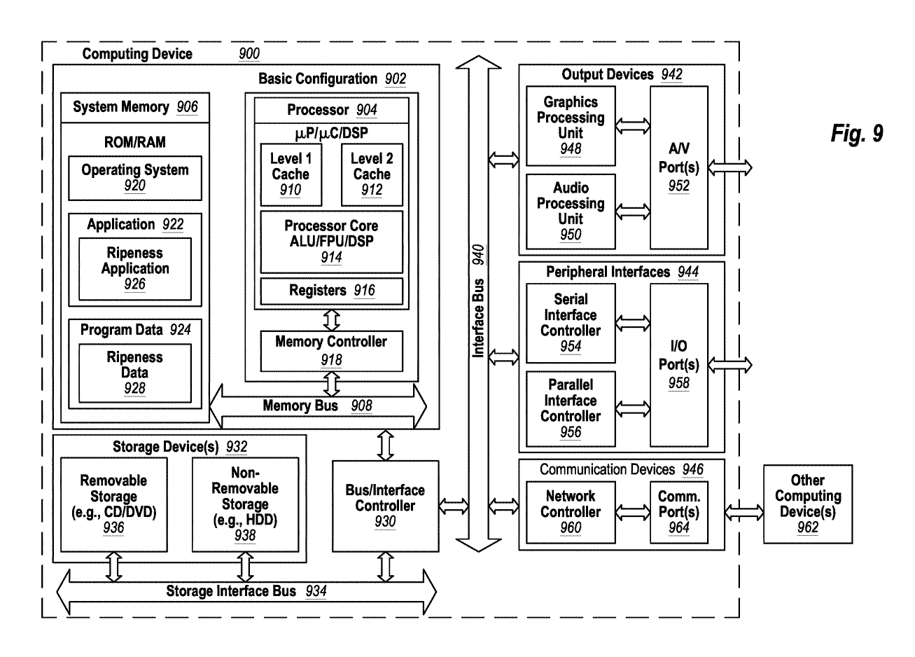

In these or other embodiments, the computing system 104 may be configured to output a value that corresponds to the output signal and that value may be compared manually with a ripeness that most closely corresponds to the value. A computing system 900 described below with respect to FIG. 9 may be an example embodiment of the computing system 104.

Modifications, additions, or omissions may be made to FIG. 1 without departing from the scope of the present disclosure. For example, the system 100 may include any number of other components not expressly illustrated or described. Further, specific implementations of the measurement circuit 102, the computing system 104, and the contact system 106 may vary.

FIG. 2 illustrates a block diagram of an example measurement circuit 202, arranged in accordance with at least some embodiments described in the present disclosure. The measurement circuit 202 may be an example of the measurement circuit 102 of FIG. 1. In the illustrated embodiment, the measurement circuit 202 may include an input circuit 204, a sensing circuit 206, a comparison circuit 208, and an output circuit 210.

The input circuit 204 may include any suitable circuit configuration configured to obtain an electrical signal as an input signal. In some embodiments, the input circuit 204 may include a signal generator configured to generate the input signal such that the input circuit 204 may obtain the input signal by generating the input signal. Additionally or alternatively, the input circuit 204 may be configured to receive the input signal from a signal generator such that the input circuit 204 may obtain the input signal by receiving the input signal.

In some embodiments, the input signal may include an Alternating Current (AC) signal with a peak-to-peak voltage that is based on a device-under-test whose electrical properties may be measured by the measurement circuit 202. For example, in some embodiments, the device-under-test may include a produce item and the peak-to-peak voltage may be selected such that adverse damage to the produce item may be reduced, negligible, or non-existent. In particular, in some embodiments, the peak-to-peak voltage of the input signal may be between 0.25 volts and 2 volts.

Additionally or alternatively, the input signal may have a frequency that may be based on the device-under-test. For example, as mentioned above and described in further detail below, in some embodiments a capacitance of the device under test may be measured between contact points of the measurement circuit 202. The frequency of the input signal may be based on a capacitance range of the device-under-test in which the frequency may be lower in instances when capacitance values within the range being relatively small and in which the frequency may be higher in instances when capacitance values within the range are relatively large. As an example, in an instance in which the capacitance values are around 20 picofarads, a frequency around or greater than 100 kilohertz may be used.

In some embodiments, the input circuit 204 may be configured to apply a gain to the input signal before the input signal is applied to the device-under-test such that the voltage of the input signal may change. The gain may be equal to or approximately equal to, 1, less than 1 or greater than 1. In some embodiments, the value of the gain may be based on the peak-to-peak voltage of in the input signal and a target peak-to-peak voltage that may be applied at the device-under-test. For example, the target peak-to-peak voltage may be 1 volt and the peak-to-peak voltage of the input signal may be 0.5 volts such that the gain may be approximately equal to or equal to 2. As another example, the target peak-to-peak voltage may be 1 volt and the peak-to-peak voltage of the input signal may be 1 volt such that the gain may be approximately equal to or equal to 1. As another example, the target peak-to-peak voltage may be 1 volt and the peak-to-peak voltage of the input signal may be 2 volts such that the gain may be approximately equal to or equal to 0.5.

In some embodiments, the input circuit 204 may include one or more amplifiers configured to apply the gain. Additionally or alternatively, the amplifiers may have a particular bandwidth. As such, in some embodiments, the frequency of the input signal may be based on a bandwidth of the amplifiers. In these or other embodiments, the input circuit 204 may include a transformer configured to change the voltage of the input signal such that the transformer may apply the gain. The input signal with the gain applied may be referred to as an “amplified input signal” in the present disclosure. The term “amplified input signal” may apply in instances where a gain of 1, less than 1, or 1 may be applied. The input circuit 204 may be configured to output the input signal or an amplified input signal as an input circuit output.

The sensing circuit 206 may be electrically coupled to the input circuit 204 and may be configured to receive the input circuit output. The sensing circuit 206 may be configured to generate a reference signal based on the input circuit output such that the reference signal may be generated based on the input signal. Additionally, the sensing circuit 206 may be configured to generate a device-under-test signal based on the input circuit output such that the device-under-test signal may be generated based on the input signal.

In these or other embodiments, the sensing circuit 206 may include a first contact point and a second contact point electrically coupled to a first contact mechanism and a second contact mechanism, respectively, of a contact system such as the contact system 106 of FIG. 1. In some embodiments, the sensing circuit 206 may be configured to generate the device-under-test signal based on a capacitance between the first contact point and the second contact point. The capacitance between the first contact point and the second contact point may be an electrical property that may indicate an electrical impedance of the device-under-test and may be based on a ripeness of the device-under-test in instances in which the device-under-test includes a produce item.

The comparison circuit 208 may be electrically coupled to the sensing circuit 206 and may be configured to receive the reference signal and the device-under-test-signal that may be generated by the sensing circuit 206. In some embodiments, the comparison circuit 208 may be configured to compare the reference signal and the device-under-test signal. In these or other embodiments, the comparison circuit 208 may be configured to generate a comparison signal based on the comparison between the reference signal and the device-under-test signal. In some embodiments, the comparison circuit 208 may be configured to determine a voltage difference between the reference signal and the device-under-test signal. In these or other embodiments, the comparison circuit 208 may be configured to output the difference as the comparison signal.

The output circuit 210 may be electrically coupled to the comparison circuit 208. The output circuit 210 may be configured to receive the comparison signal and emit an output signal based on the comparison signal. In some embodiments, the comparison signal may include an AC signal and the output circuit 210 may be configured to convert the comparison signal from an AC signal to a DC signal. In these or other embodiments, the DC signal converted from the AC comparison signal may be used as the output signal. Additionally or alternatively, the output circuit 210 may be configured to apply a gain to the DC signal and the amplified DC signal may be used as the output signal. In the present disclosure, the term “amplified DC signal” may refer to a resulting signal after a gain of 1, less than 1, or greater than 1 has been applied to the DC signal converted from the AC comparison signal.

The output signal that may be emitted by the output circuit 210 may be based on the comparison signal, which may be based on the device-under-test signal, which may be based on the electrical impedance (e.g., as indicated by capacitance) of the device-under-test, which may indicate a ripeness of the device-under-test in instances in which the device-under-test includes a produce item. As such, the output signal may indicate a ripeness of a produce item in some embodiments.

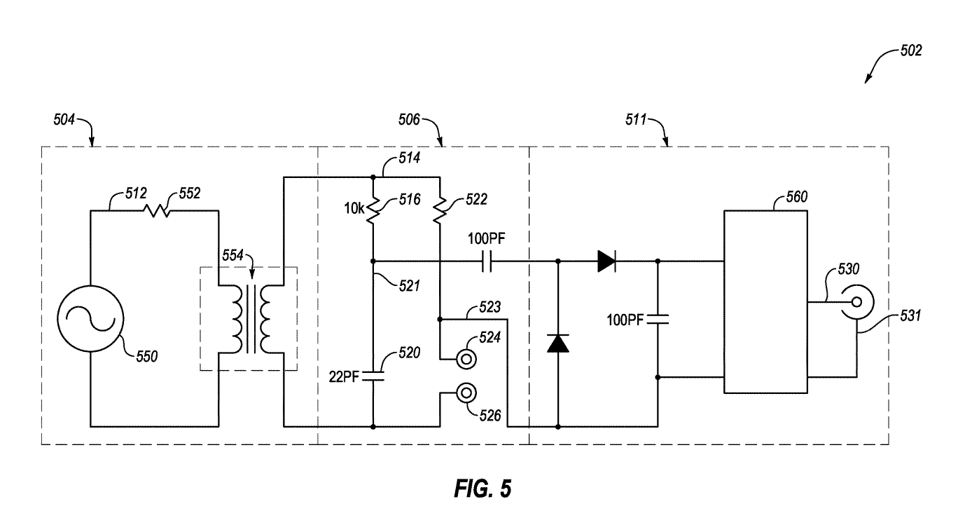

Modifications, additions, or omissions may be made to FIG. 2 without departing from the scope of the present disclosure. For example, the measurement circuit 200 may include any number of other components not expressly illustrated or described. Further, one or more of the circuits described above may be configured differently or omitted from the measurement circuit 202. For example, in some embodiments, the comparison circuit 208 and the output circuit 210 may be configured such that their described functionality may be performed by the same circuitry, such as described with respect to an example embodiment of a measurement circuit 502 described with respect to FIG. 5.

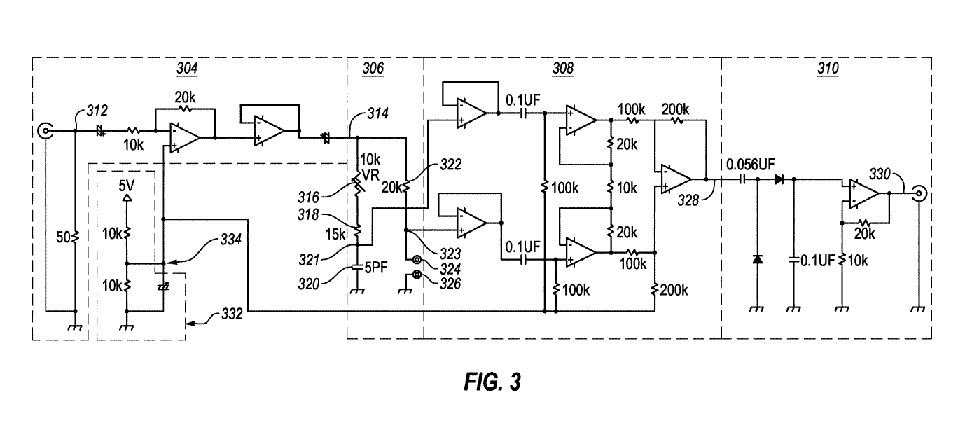

FIG. 3 illustrates an example measurement circuit 302, arranged in accordance with at least some embodiments described in the present disclosure. The measurement circuit 302 may be an example embodiment of the measurement circuit 202 of FIG. 2. The measurement circuit 302 may include an input circuit 304, a sensing circuit 306, a comparison circuit 308, and an output circuit 310.

The input circuit 304 may be an example embodiment of the input circuit 204 of FIG. 2. The input circuit 304 may be electrically coupled to a node 312 of the measurement circuit 302. The input circuit 304 may be configured to receive an input signal at the node 312. The input signal may be analogous to the input signal described above with respect to FIG. 2. The input circuit 304 may include multiple elements such as resistors, capacitors, operational amplifiers (“op-amps”), etc. configured in the manner illustrated. The illustrated configuration may be such that the input circuit 304 may apply a gain to the input signal received at the node 312 to generate an amplified input signal that may be output as an input circuit output at a node 314 of the measurement circuit 302. The specific configuration of the input circuit 304 and the values illustrated are examples and are not meant to be limiting. For example, in some embodiments, the input circuit 304 may include a signal generator configured to generate the input signal.

The sensing circuit 306 may be coupled to the node 314 such that the sensing circuit 306 may receive the input circuit output that may be output by the input circuit 304. The sensing circuit 306 may include reference circuitry and device-under-test circuitry that may be coupled to the node 314. The reference circuitry may be configured to generate a reference signal based on the input circuit output and the device-under-test circuitry may be configured to generate a device-under-test signal based on the input circuit output and a capacitance of the device-under-test.

In particular, in some embodiments, the device-under-test circuitry may include a device-under-test resistive element 322, a device-under-test node 323, a first contact point 324, and a second contact point 326. The device-under-test resistive element 322 may be coupled between the node 314 and the device-under-test node 323. The device-under-test resistive element 322 may include any suitable device that may act as a resistor.

The first contact point 324 may be coupled to the device-under-test node 323 also. Further, the second contact point 326 may be coupled to ground. Further, the first contact point 324 may be electrically coupled to a first contact mechanism of a contact system and the second contact point 326 may be electrically coupled to a second contact mechanism of the contact system. As described above, in some embodiments, the contact mechanisms may be configured to contact a produce item such that an electrical property between the first contact point 324 and the second contact point 326 may vary depending on ripeness of the produce item. In some instances, the electrical property may include electrical impedance that may be indicated by capacitance between the first contact point 324 and the second contact point 326. In some embodiments, the amount of resistance of the device-under-test resistive element 322 may be based on a range of the electrical impedance that may be between the first contact point 324 and the second contact point 326 that may correspond to the produce item.

The resistance of the device-under-test resistive element 322 and the capacitance between the first contact point 324 and the second contact point 326 may generate a capacitive-resistive (“CR”) oscillator. The CR oscillator may cause a device-under-test signal to be output at the device-under-test node 323 based on the input circuit output received at the node 314 and based on the capacitance between the first contact point and the second contact point. As indicated above, in some embodiments, the capacitance may vary depending on ripeness of the device-under-test in instances in which the device-under-test includes a produce item. As such, the device-under-test signal may be based on ripeness of a produce item in some embodiments.

Additionally, in some embodiments, the reference circuitry may include a first reference-resistive element 316, a second reference-resistive element 318, a reference node 321, and a capacitive element 320. The first reference-resistive element 316 may be coupled between the node 314 and the second reference-resistive element 318. Further, the second reference-resistive element 318 may be coupled between the first reference-resistive element 316 and a reference node 321. In addition, the capacitive element 320 may be coupled between the reference node 321 and ground.

The resistance of the reference-resistive elements 316 and 318 and the capacitance of the capacitive element 320 may also generate a CR oscillator. The resulting CR oscillator may cause a reference signal to be output at the reference node 321 based on the input circuit output received at the node 314. In some embodiments, the combined resistance of the reference-resistive elements 316 and 318 may be equal to or approximately equal to the resistance of the device-under-test resistive element 322. In some embodiments, the approximation may be such that the combined resistance of the reference-resistive elements 316 and 318 may be within ±25% of the resistance of the device-under-test resistive element 322.

In some embodiments, the first reference-resistive element 316 and the second reference-resistive element 318 may include any suitable device that may act as a resistor. In some embodiments, the first reference-resistive element 316 or the second reference-resistive element 318 may include any suitable device that may act as a variable resistor. In the illustrated example, the first reference-resistive element 316 is depicted as a variable resistor, but the second reference-resistive element could also or alternatively be a variable resistor. In some embodiments, the resistance of the variable resistor may be modified depending on different produce items that may be used as the device-under test because of differences in capacitances between different produce items. In these or other embodiments, a single resistive element may be used as the first reference-resistive element 316 and the second reference-resistive element 318.

Additionally or alternatively, in some embodiments, a resistor or capacitor may be used as the capacitive element 320. However, using a capacitor may help in instances in which the frequency of the input circuit output is relatively unstable because variations in electrical impedance of a capacitor due to frequency changes may be relatively similar to variations in electrical impedance between the first contact point 324 and the second contact point 326. The specific configuration of the sensing circuit 306 and the values illustrated are examples and are not meant to be limiting.

The comparison circuit 308 may be an example of the comparison circuit 208 of FIG. 2. The comparison circuit 308 may be coupled to the reference node 321 and the device-under-test node 323. As such, the comparison circuit 308 may be configured to receive the reference signal at the reference node 321 and to receive the device-under-test signal at the device-under-test node 323. The comparison circuit 308 may be configured to compare the reference signal and the device-under-test signal. Based on the comparison, the comparison circuit 308 may be configured to output a comparison signal at a node 328 of the measurement circuit 302. As indicated above, in some embodiments, the comparison signal may indicate a difference between the reference signal and the device-under-test signal. As mentioned above, the value of the device-under-test signal may vary depending on ripeness of the device-under-test in instances in which the device-under-test includes a produce item. As such, the difference between the reference signal and the device-under-test signal may vary depending on ripeness of a produce item such that the comparison signal may be based on the ripeness. In some embodiments, the comparison circuit 308 may include multiple elements such as resistors, capacitors, op-amps etc. configured in the manner illustrated to generate the comparison signal based on the reference signal and the device-under-test signal. The specific configuration of the comparison circuit 308 and the values illustrated are examples and are not meant to be limiting.

The output circuit 310 may be an example of the output circuit 210 of FIG. 2. The output circuit 310 may be coupled to the node 328 such that the output circuit 310 may receive the comparison signal that may be generated by the comparison circuit 308. The output circuit 310 may be configured to convert the comparison signal from an AC signal to a DC signal. Additionally or alternatively, the output circuit 310 may be configured to apply a gain to the DC signal. In these or other embodiments, the output circuit 310 may include a low-pass filter configured to filter out noise that may be with the DC signal. The output circuit 310 may be configured to emit the DC signal as an output signal at a node 330 of the measurement circuit 302. In some embodiments, the output circuit 310 may include multiple elements such as resistors, capacitors, op-amps, diodes, etc. configured in the manner illustrated to generate the output signal at the node 330 based on the comparison signal received at the node 328. The specific configuration of the output circuit 310 and the values illustrated are examples and are not meant to be limiting.

In some embodiments, the output signal may include a voltage at the node 330, which may vary depending on the comparison signal, which as indicated above may vary based on ripeness of a produce item of which contact mechanisms electrically coupled to the first and second contact points 324 and 326 may be in contact. As such, the output signal may indicate ripeness of the produce item in some embodiments.

In some embodiments, the measurement circuit 302 may include a biasing circuit 332. The biasing circuit 332 may be configured to set a biasing voltage at a biasing node 334. The biasing node 334 may be coupled to the input circuit 304 and the comparison circuit as illustrated such that one or more elements of the input circuit 304 or the comparison circuit 308 may be biased according to the biasing voltage at the biasing node 334. In some embodiments, the biasing circuit 332 may be configured such that the biasing voltage at the biasing node 334 may be set such that the voltages of the AC signals within the measurement circuit 302 may be positive even in light of the voltage swings of the AC signals. In the illustrated example, the biasing circuit 332 may be configured to set a biasing voltage of 2.5 volts at the biasing node 334. The specific configuration of the biasing circuit 332 and the values illustrated are examples and are not meant to be limiting.

Modifications, additions, or omissions may be made to the measurement circuit 302 without departing from the scope of the present disclosure. For example, as indicated above, the specific configuration of the circuits of the measurement circuit 302 and the values illustrated are examples and are not meant to be limiting. In particular, elements may be added to or removed from the measurement circuit 302. Additionally, the specific couplings and placement of elements may vary depending on implementations.

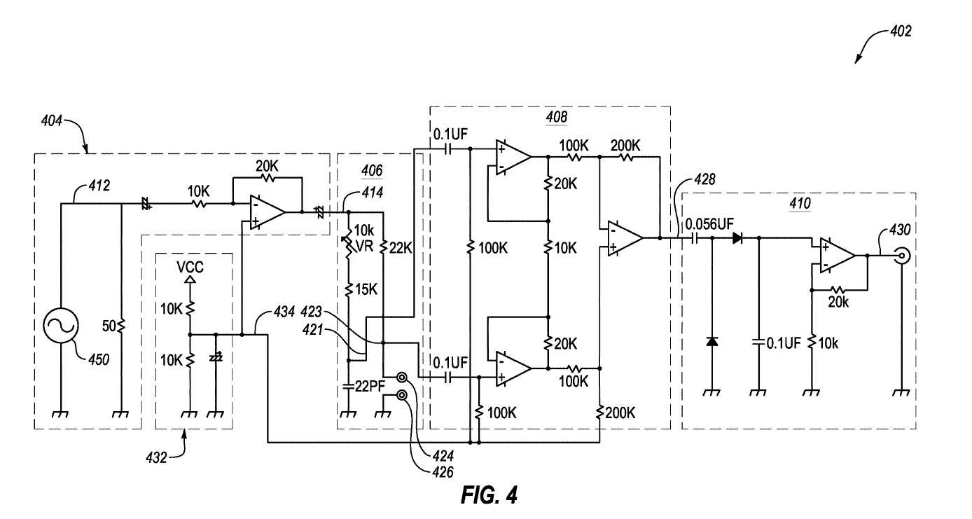

FIG. 4 illustrates an example measurement circuit 402, arranged in accordance with at least some embodiments described in the present disclosure. The measurement circuit 402 may be an example embodiment of the measurement circuit 202 of FIG. 2. The measurement circuit 402 may include an input circuit 404, a sensing circuit 406, a comparison circuit 408, and an output circuit 410.

The input circuit 404 may be another example embodiment of the input circuit 204 of FIG. 2. In the illustrated embodiment, the input circuit 404 may include a signal generator 450 configured to generate an input signal that may be received at an input node 412. The input signal may be analogous to the input signal described above with respect to FIG. 2. The input circuit 404 may include multiple elements such as resistors, capacitors, operational amplifiers (“op-amps”), etc. configured in the manner illustrated. The illustrated configuration may be such that the input circuit 404 may apply a gain to the input signal received at the input node 412 to generate an amplified input signal that may be output as an input circuit output at a node 414 of the measurement circuit 402. In some embodiments, the input circuit 404 may be configured to have a relatively low impedance at the node 414. The specific configuration of the input circuit 404 and the values illustrated are examples and are not meant to be limiting. Further, in some embodiments, the input circuit 404 may not include the signal generator 450.

The sensing circuit 406 may be coupled to the node 414 such that the sensing circuit 406 may receive the input circuit output that may be output by the input circuit 404. The sensing circuit 406 may be configured analogously to the sensing circuit 306 of FIG. 3. Similar to the sensing circuit 306, the sensing circuit 406 may be configured to generate a reference signal at a reference node 421 of the measurement circuit 402 and may be configured to generate a device-under-test signal at a device-under-test node 423 of the measurement circuit 402. The specific configuration of the sensing circuit 406 and the values illustrated are examples and are not meant to be limiting.

The comparison circuit 408 is another example of the comparison circuit 208 of FIG. 2. The comparison circuit 408 may be coupled to the reference node 421 and the device-under-test node 423. As such, the comparison circuit 408 may be configured to receive the reference signal at the reference node 421 and to receive the device-under-test signal at the device-under-test node 423. The comparison circuit 408 may be configured to compare the reference signal and the device-under-test signal. Based on the comparison, the comparison circuit 408 may be configured to output a comparison signal at a node 428 of the measurement circuit 402. The comparison signal may indicate a difference between the reference signal and the device-under-test signal similar to the comparison signal discussed above with respect to the comparison circuit 308. In some embodiments, the comparison circuit 408 may include multiple elements such as resistors, capacitors, op-amps etc. configured in the manner illustrated to generate the comparison signal based on the reference signal and the device-under-test signal. The specific configuration of the comparison circuit 408 and the values illustrated are examples and are not meant to be limiting.

The output circuit 410 may be another example of the output circuit 210 of FIG. 2. The output circuit 410 may be coupled to the node 428 such that the output circuit 410 may receive the comparison signal that may be generated by the comparison circuit 408. The output circuit 410 may be configured to convert the comparison signal from an AC signal to a DC signal. Additionally or alternatively, the output circuit 410 may be configured to apply a gain to the DC signal. In these or other embodiments, the output circuit 410 may include a low-pass filter configured to filter out noise that may be with the DC signal. The output circuit 410 may be configured to emit the DC signal as an output signal at a node 430 of the measurement circuit 402. In some embodiments, the output circuit 410 may include multiple elements such as resistors, capacitors, op-amps, diodes, etc. configured in the manner illustrated to generate the output signal at the node 430 based on the comparison signal received at the node 428. The specific configuration of the output circuit 410 and the values illustrated are examples and are not meant to be limiting.

In some embodiments, the output signal may include a voltage at the node 430, which may vary depending on the comparison signal, which as indicated above may vary based on ripeness of a produce item of which contact mechanisms electrically coupled to first and second contact points 424 and 426 of the sensing circuit 406 may be in contact. As such, the output signal may indicate ripeness of the produce item in some embodiments.

In some embodiments, the measurement circuit 402 may include a biasing circuit 432 that may be configured to set a biasing voltage at a biasing node 434. The biasing circuit 432 and the biasing voltage may be analogous to the biasing circuit 332 and associated biasing voltage described above with respect to FIG. 3.

Modifications, additions, or omissions may be made to the measurement circuit 402 without departing from the scope of the present disclosure. For example, as indicated above, the specific configuration of the circuits of the measurement circuit 402 and the values illustrated are examples and are not meant to be limiting. In particular, elements may be added to or removed from the measurement circuit 402. Additionally, the specific couplings and placement of elements may vary depending on implementations.

FIG. 5 illustrates an example measurement circuit 502, arranged in accordance with at least some embodiments described in the present disclosure. The measurement circuit 502 may be another example embodiment of the measurement circuit 202 of FIG. 2. The measurement circuit 502 may include an input circuit 504, a sensing circuit 506, and a combined comparison/output circuit 511 (“combination circuit 511”).

The input circuit 504 may be an example embodiment of the input circuit 204 of FIG. 2. In the illustrated embodiment, the input circuit 504 may include a signal generator 550 configured to generate an input signal that may be received at an input node 512. The input signal may be analogous to the input signal described above with respect to FIG. 2. The input circuit 504 may include a resistive element 552 and a transformer 554 configured in the manner illustrated. The illustrated configuration may be such that the transformer may apply change the voltage of the input signal received at the input node 512 to generate a changed input signal that may be output as an input circuit output at a node 514 of the measurement circuit 502. In the present disclosure, the change in the voltage of the input signal performed by the transformer may be included in the term “gain” and the changed input signal may be included in the term “amplified input signal” although such terms are typically used with respect to amplifiers and not transformers. In some embodiments, the input circuit 504 may be configured to have a relatively low impedance at the node 514. The specific configuration of the input circuit 504 and the values illustrated are examples and are not meant to be limiting. Further, in some embodiments, the input circuit 504 may not include the signal generator 550.

The sensing circuit 506 may be coupled to the node 514 such that the sensing circuit 506 may receive the input circuit output that may be output by the input circuit 504. The sensing circuit 506 may include reference circuitry and device-under-test circuitry that may be coupled to the node 514. The reference circuitry may be configured to generate a reference signal based on the input circuit output and the device-under-test circuitry may be configured to generate a device-under-test signal based on the input circuit output and a capacitance of the device-under-test.

In particular, in some embodiments, the device-under-test circuitry may include a device-under-test resistive element 522, a device-under-test node 523, a first contact point 524, and a second contact point 526 that may be analogous to the device-under-test resistive element 322, the device-under-test node 323, the first contact point 324, and a second contact point 326, respectively, of the sensing circuit 306 of FIG. 3. The device-under-test circuitry may accordingly be configured to generate the device-under-test signal that may be output at the device-under-test node 523.

Additionally, in some embodiments, the reference circuitry may include resistive element 516, a reference node 521, and a capacitive element 520 configured as illustrated. The reference circuitry may be configured to generate the reference signal that may be output at the reference node 521. The specific configuration of the sensing circuit 506 and the values illustrated are examples and are not meant to be limiting.

The combination circuit 511 may be an example of a combination of the comparison circuit 208 and the output circuit 210 of FIG. 2. The combination circuit 511 may be coupled to the reference node 521 and the device-under-test node 523. The combination circuit 311 may be configured to convert the reference signal and the device-under-test signal from AC signals to DC signals. In the illustrated embodiment, the combination circuit 311 may include diodes and a capacitor configured as illustrated to convert the reference signal and the device-under-test signal from AC signals to DC signals.

In these or other embodiments, the combination circuit 511 may include a low-pass filter 560 configured to receive the DC reference signal and the DC device-under-test signal. The low-pass filter 560 may be configured to filter out noise that may be included in the DC signals.

The combination circuit 511 may include a first output node 530 and a second output node 531. The first output node 530 may be configured to output the filtered DC reference signal in the form of a voltage at the first output node 530. The second output node 531 may be configured to output the filtered DC device-under-test signal in the form of a voltage at the second output node 531. A difference between the voltage at the first output node 530 and the voltage at the second output node 531 may indicate a difference between the device-under-test signal and the device-under-test signal. The difference in voltages between the first output node 530 and the second output node 531 may be a comparison signal that may also be an output signal. As mentioned above, the difference between the reference signal and the device-under-test signal may indicate a ripeness of the device-under-test in instances in which the device-under-test includes a produce item. As such, the combination circuit 511 may be configured to generate an output signal based on a comparison signal in which the output signal may indicate ripeness of a produce item. The specific configuration of the combination circuit 511 and the values illustrated are examples and are not meant to be limiting.

Modifications, additions, or omissions may be made to the measurement circuit 502 without departing from the scope of the present disclosure. For example, as indicated above, the specific configuration of the circuits of the measurement circuit 502 and the values illustrated are examples and are not meant to be limiting. In particular, elements may be added to or removed from the measurement circuit 502. Additionally, the specific couplings and placement of elements may vary depending on implementations.

FIG. 6A illustrates an example contact system 606, arranged in accordance with at least one embodiment of the present disclosure. The contact system 606 may be configured to contact a produce item 650 such that a ripeness of the produce item 650 may be determined based on an electrical property of the produce item 650. The produce item may include any suitable fruit or vegetable whose ripeness may be determined based on one or more of its electrical properties. The contact system 606 may include a first contact mechanism 652, a second contact mechanism 654, a first roller 656a, a second roller 656b, a motor 658, a rotatable shaft 664, a first wire 660, and a second wire 662.

The first roller 656a and the second roller 656b may be configured such that the first roller 656a and the second roller 656b may support the produce item 650. In some embodiments, the rollers 656 may coupled to the rotatable shaft 664 such that the rollers 656 may rotate in response to rotation of the rotatable shaft 664. Further, in some embodiments, the rollers 656 may include one or more rings 668 configured to interact with the produce item 650. The rings 668 may include any suitable material that may interact with the produce item 650 in a manner that may not damage the produce item 650. Additionally or alternatively, the rings 668 may be such that a sufficient amount of friction may be between the rings 668 and the produce item 650 such that rotation of the rollers 656 may cause the produce item 650 to rotate. In some embodiments, the rings 668 may include a rubber material or something else similar to rubber in its properties.

A spacing between and sizing of the rollers 656 may be based on a type of produce item for which the contact system 606 may be configured. For example, the sizing and spacing may be based on an average size of the type of the produce item 650. Additionally or alternatively, the selection of material for the rings 668 may be based on surface properties of the type of produce item for which the contact system 606 may be configured. For example, the material for the rings 668 may be softer for produce items that may bruise or damage relatively easily and may be harder for produce items that may not bruise or damage relatively easily.

The motor 658 may include any suitable system, apparatus, or device configured to generate a rotational force. Further, the motor 658 may be coupled to the rotatable shaft 664 such that the rotational force generated by the motor 658 may rotate the rotatable shaft 664. As such, the motor 658 may be configured to rotate the produce item 650.

The first contact mechanism 652 may be positioned within the contact system 606 such that it may contact the produce item 650 at a first contact area 666 of an outside surface of the produce item 650. In some embodiments, the first contact mechanism 652 may be configured to move along the produce item 650 as the produce item 650 is rotating as caused by the motor 658, the rotatable shaft 664, and the rollers 656. For example, the first contact mechanism 652 may be configured to move along the produce item 650 in the direction of an arrow 670 illustrated in FIG. 6A. As such, in some embodiments, a location of the first contact area 666 may vary as the first contact mechanism 652 may move along the produce item 650.

The first contact mechanism 652 may include a roller 672 and one or more other rings 674. The roller 672 may include at least an electrically conductive (e.g., metallic) surface in some embodiments. In the illustrated embodiment, the first contact mechanism 652 may include three rings 674, but this is merely given as an example. The rings 674 may be made of a non-conductive material such as rubber such that they may be insulative. Further, the rings 674 may be configured and placed around the roller 672 such that the rings 674 may have a raised profile with respect to a surface of the roller 672. The raised profile may be such that a gap between the surface of the produce item 650 and the surface of the roller 672 may be present at the first contact area 666.

In some embodiments, a size of the gap between the surface of the produce item 650 and the surface of the roller 672 may vary depending on a hardness of the produce item 650. As indicated above, the hardness of the produce item 650 may vary depending on the ripeness of the produce item 650 such that the size of the gap may also vary based on the ripeness of the produce item 650.

For example, FIG. 6B includes an example 680 that illustrates a first gap “Gap1” between the surface of the roller 672 and the surface of the produce item 650 at the first contact area 666. Further, FIG. 6B also includes an example 682 that illustrates a second gap “Gap2” between the surface of the roller 672 and the surface of the produce item 650 at the first contact area 666. As illustrated in FIG. 6B, the first gap may be greater than the second gap. The first gap may be greater than the second gap based on the produce item 650 being harder in the example 680 than in the example 682. Also, a size of the first contact area 666 in contact with the first contact mechanism (e.g., a surface area of contact with the rings 674) may be greater in the example 682 as opposed to the example 680 based on the produce item 650 being softer in the example 682 than in the example 680.

Returning to FIG. 6A, the second contact mechanism 654 may be positioned within the contact system 606 such that it may contact the produce item 650 at a second contact area 667 of the outside surface of the produce item 650. In the illustrated example, the second contact mechanism 654 may include a roller 676. The roller 676 may include at least an electrically conductive (e.g., metallic) surface in some embodiments. In these or other embodiments, the second contact mechanism 654 may include one or more rings analogous to the rings 674. In the illustrated example, the second contact mechanism 654 may be placed and configured to support at least a portion of the produce item 650 such that a weight of the produce item 650 presses the surface of the produce item 650 against the surface of the roller 676.

In some embodiments, a size of the second contact area in contact with the second contact mechanism 654 may vary depending on a hardness of the produce item 650. For example, FIG. 6C includes an example 690 that illustrates a first size of the second contact area 667 of the surface of the produce item 650 in contact with the surface of the roller 676. FIG. 6C also includes an example 692 that illustrates a second size of the second contact area 667 of the surface of the produce item 650 in contact with the surface of the roller 676. As illustrated in FIG. 6C, the first size may be smaller than the second size. The first size may be smaller than the second size based on the produce item 650 being harder in the example 690 than in the example 692.

Returning to FIG. 6A, the surface of the roller 672 may be electrically coupled to a first wire 660. The first wire 660 may be electrically coupled to a first contact point of a measurement circuit, such as the measurement circuits described above. Additionally, the surface of the roller 676 may be electrically coupled to a second wire 662. The second wire 662 may be electrically coupled to a second contact point of a measurement circuit, such as the measurement circuits described above. As indicated above, electrical impedance of the produce item 650 between the first contact mechanism 652 and the second contact mechanism 654 may indicate a ripeness of the produce item 650.

Further, an indication of the electrical impedance (e.g., capacitance) may vary depending on the size of the gap between the surface of the roller 672 and the surface of the produce item 650 at the first contact area 666. Additionally or alternatively, an indication of the electrical impedance (e.g., the capacitance) may vary depending on the size of the second contact area 667 in contact with the surface of the roller 676. As indicated above, the size of the gap and the size of the contact area may vary depending on ripeness of the produce item 650 such that the capacitance that may be caused by changes in the size of the gap or size of the contact area may indicate ripeness of the produce item 650.

As such, the contact system 606 may be configured to interface with the produce item 650 such that a ripeness of the produce item 650 may be determined based on electrical impedance of the produce item 650. Modifications, additions, or omissions may be made to the contact system 606 without departing from the scope of the present disclosure.

For example, in some embodiments, the first contact mechanism 652 may not include the rings 674 or the second contact mechanism 654 may include the rings 674. Further, in some embodiments, the first contact mechanism 652 may be may be placed and configured to support at least a portion of the produce item 650 such that a weight of the produce item 650 presses the surface of the produce item 650 against the surface of the roller 672 or against the rings 674. In these or other embodiments, the second contact mechanism 654 may be configured to move along the produce item 650 similar to the first contact mechanism 652.

FIG. 7A illustrates an example contact system 702, arranged in accordance with at least one embodiment of the present disclosure. The contact system 702 may be configured to contact a produce item such that a ripeness of the produce item may be determined based on an electrical property of the produce item. The produce item may include any suitable fruit or vegetable whose ripeness may be determined based on one or more of its electrical properties (e.g, electrical impedance as indicated by capacitance). The contact system 702 may include a first contact mechanism 752, second contact mechanisms 754a and 754b, and a guard ring 756.

The guard ring 756 may include any suitable insulative material and may be configured to contact a surface of the produce item. The guard ring 756 may include a rubber or silicone material in some embodiments. The guard ring 756 may be configured such that it may provide a substantially non-destructive interface between the contact system 702 and the surface of the produce item.

The first contact mechanism 752 may be disposed within an opening in the guard ring 756. In some embodiments, the first contact mechanism 752 may have at least a portion thereof that is generally spherical. Additionally or alternatively, the first contact mechanism may include a generally spherical element such as a ball. Further, in some embodiments, at least a surface of the first contact mechanism 752 may be electrically conductive. For example, in some embodiments, the first contact mechanism 752 may include a steel ball. In some embodiments, the size of the steel ball may be selected based on the type of produce item such that little to no damage may occur to the produce item when the steel ball is pressed against the produce item as discussed below. By way of example, in some embodiments, a diameter of the steel ball may be between 2.5 mm and 20 mm.

In some embodiments, the contact system 702 may be configured such that the first contact mechanism 752 may contact a first contact area of a surface of the produce item. Further, the contact system 702 may be configured such that the first contact mechanism 752 may press against the surface of the produce item to apply a force to the surface of the produce item. The force may be such that damage to the produce item may be negligible or non-existent. The force may also be such that a size of the first contact area in contact with the first contact mechanism 752 may change according to a hardness of the produce item. An example amount of force that may be used is 30-300 grams per centimeter squared. The change in the size of the first contact area is illustrated and detailed further below with respect to FIG. 7B.

FIG. 7A also illustrates example components that may be included with the contact system 702 to apply force to the surface of the produce item by the first contact mechanism 752. For example, in some embodiments, the first contact mechanism 752 may be coupled to a plunger 758 in the manner illustrated. The contact system 702 may include any suitable system, apparatus, or device, configured to apply a deployment force that runs parallel to a length of the plunger 758 in a direction toward the first contact mechanism 752. As such, when the contact system 702 is oriented such that the guard ring 756 is pressed against the surface of the produce item, the deployment force applied to the plunger 758 may also press the first contact mechanism 752 against the surface of the produce item.

In some embodiments, the plunger 758 may include a magnetic portion 759 (or may be coupled to a magnet) and the contact mechanism may include an electric coil 757 that may be configured to produce the deployment force. In particular, the electric coil 757 may be positioned around the magnetic portion 758. Additionally, the electric coil 757 may be configured to receive an electric current that may produce a magnetic field that may interact with the magnetic field of the magnetic portion 759 to produce the deployment force.

In some embodiments, the contact system 702 may also include a biasing mechanism 760. The biasing mechanism 760 may include any suitable system, apparatus, or device configured to apply a retracting force to the first contact mechanism 752. The retracting force may be opposite the deployment force such that it may retract the first contact mechanism 752 away from the surface of the produce item. In the illustrated example, the biasing mechanism 760 may include a spring that may be coupled to the plunger 758 such that the spring may compress in response to the deployment force being applied to the plunger 758. As such, in response to the deployment force being removed, the spring may expand and the expansion force of the spring may produce the retracting force to retract the first contact mechanism. In some embodiments, the deployment force may be such that it may overcome the retracting force of the biasing mechanism 760 and such that the first contact mechanism 752 may apply a target amount of force to the surface of the produce item. In some embodiments, the biasing mechanism 760 may be omitted and the electric coil 757 may be configured to receive an electric current that may produce a magnetic field that may interact with the magnetic field of the magnetic portion 759 to produce the retracting force.

FIG. 7B includes an example 770 that illustrates a first size of a contact area 761 of the surface of a produce item 750 in contact with the first contact mechanism 752. FIG. 7C also includes an example 772 that illustrates a second size of the contact area 761 of the surface of the produce item 750 in contact with the first contact mechanism 752. In addition, FIG. 7B includes an example 774 that illustrates a third size of the contact area 761 of the surface of the produce item 750 in contact with the first contact mechanism 752. In the example 770 the produce item 750 may be softer than the produce item 750 in the example 772 and the produce item 750 in the example 772 may be softer than the produce item in the example 774. As illustrated in FIG. 7B, a shape of the produce item 750 around the first contact mechanism 752 may vary depending on the softness of the produce item 750. The change differences in the shape may be such that the first size may be larger than the second size and the second size may be larger than the first size.

Returning to FIG. 7A, the second contact mechanisms 754a and 754b may be positioned within the contact system 702 such that they may each contact the produce item at a second contact area of the outside surface of the produce item. In the illustrated example, the second contact mechanisms 754 may each include a roller. The rollers may include at least an electrically conductive (e.g., metallic) surface in some embodiments. In the illustrated example, the second contact mechanisms 754 may be placed and configured such that a substantial amount of the produce item may be between the first contact mechanism 752 and the second contact mechanisms 754.

In some embodiments, a size of the second contact areas in contact with the second contact mechanisms 754 may vary depending on the hardness of the produce item. For example, in some embodiments, the size of a second contact area may vary depending on the hardness of the produce item such as illustrated with respect to FIG. 6C.

Returning to FIG. 7A, the surface of the first contact mechanism 752 may be electrically coupled to a first wire 764. In the illustrated embodiment, the first wire 764 may be electrically coupled to the plunger 758, which may be electrically conductive (e.g., metallic), and the first contact mechanism 752 may include a steel ball such that the first contact mechanism 752, the plunger 758, and the first wire 764 may be electrically conductive. The first wire 764 may be electrically coupled to a first contact point of a measurement circuit, such as the measurement circuits described above. Additionally, the surfaces of the second contact mechanisms 754a and 754b may be electrically coupled to second wires 762a and 762b, respectively. The second wires 762 may be coupled to a second contact point of a measurement circuit, such as the measurement circuits described above. As indicated above, electrical impedance of the produce item between the first contact mechanism 752 and the second contact mechanisms 754 may indicate a ripeness of the produce item.

Further, an indication of the electrical impedance (e.g., capacitance) may vary depending on the size of the contact areas of the first contact mechanism 752 or the second contact mechanisms 754. As indicated above, the sizes of the contact areas may vary depending on ripeness of the produce item such that the capacitance that may be caused by changes in sizes of one or more contact areas may indicate ripeness of the produce item.

As such, the contact system 702 may be configured to interface with the produce item such that a ripeness of the produce item may be determined based on one or more electrical properties of the produce item. Modifications, additions, or omissions may be made to the contact system 702 without departing from the scope of the present disclosure.

For example, in some embodiments, the sizing and spacing of the first contact mechanism 752 and the second contact mechanisms 754 may vary depending on a type of produce item for which the contact system 702 may be configured. In addition, in some embodiments, the contact system 702 may include more or fewer second contact mechanisms 754 or more first contact mechanisms 752.

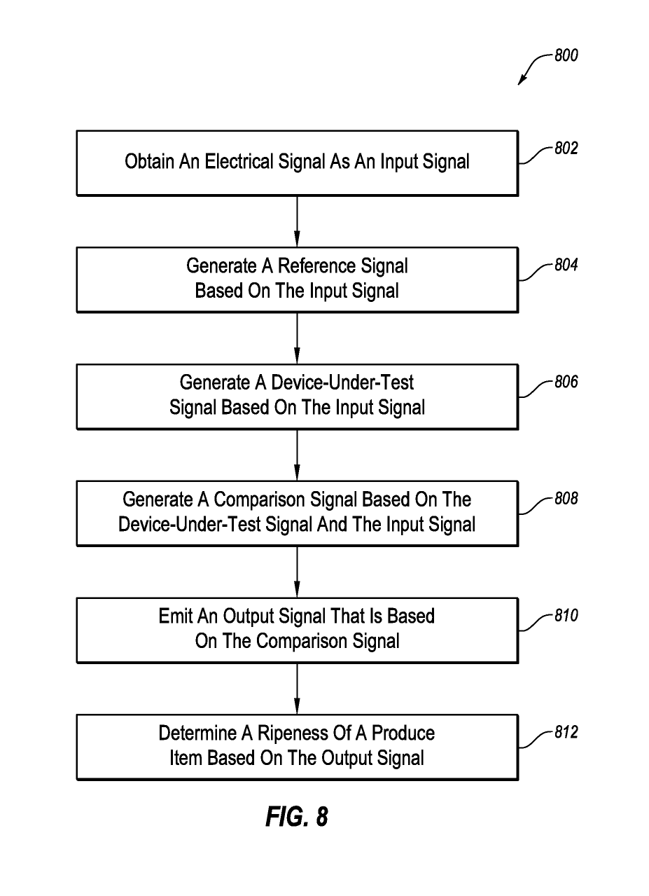

FIG. 8 illustrates a flow diagram of an example method 800 of determining a ripeness of a produce item, in accordance to one or more embodiments described in the present disclosure. The method 800 may be performed in whole or in part by one or more elements of the system 100 and its example components described in the present disclosure. The method 800 includes various operations, functions, or actions as illustrated by one or more of blocks 802, 804, 806, 808, 810, or 812.

For this and other processes and methods disclosed herein, the operations performed in the processes and methods may be implemented in differing order. Furthermore, the depicted operations are only provided as examples, and some of the operations may be optional, combined into fewer operations, supplemented with other operations, or expanded into additional operations without detracting from the essence of the disclosed embodiments. The method 800 may begin at block 802.

In block 802 (“Obtain An Electrical Signal As An Input Signal”), an electrical signal may be obtained as an input signal. In some embodiments, the input signal may be obtained by receiving the electrical signal. Additionally or alternatively, the input signal may be obtained by generating the electrical signal. In some embodiments, the input signal may be analogous to the input signal described above with respect to FIG. 1.

In block 804 (“Generate A Reference Signal Based On The Input Signal”), a reference signal may be generated based on the input signal. In some embodiments, the reference signal may be generated such as described above with respect to FIG. 3, 4, or 5. In block 806 (“Generate A Device-Under-Test Signal Based On The Input Signal”), a device-under-test signal may be generated based on the input signal. In some embodiments, the device-under-test signal may be generated such as described above with respect to FIG. 3, 4, or 5.

In block 808 (“Generate A Comparison Signal Based On The Device-Under-Test Signal And The Input Signal”), a comparison signal may be generated based on the device-under-test signal and the input signal. In some embodiments, the comparison signal may be generated such as described above with respect to FIG. 3, 4, or 5. In block 810 (“Emit An Output Signal That Is Based On The Comparison Signal”), an output signal may be emitted based on the comparison signal. In some embodiments, the output signal may be generated and emitted such as described above with respect to FIG. 3, 4, or 5. In block 812 (“Determine A Ripeness Of A Produce Item Based On The Output Signal”), a ripeness of a produce item may be determined based on the output signal. In some embodiments, the ripeness may be determined based on a relationship between the output signal and ripeness such as described above.

Modifications, additions, or omissions may be made to the method 800 without departing from the scope of the present disclosure. For example, in some embodiments, various types of circuits and implementations may be used to perform the operations of the method 800. Further, the order of the operations may be performed differently than described. Additionally or alternatively, one or more operations may be performed at the same time. Moreover, the method 800 may include more or fewer operations than those described.

FIG. 9 is a block diagram illustrating an example computing system 900 that is arranged to direct one or more operations associated with determining ripeness of a produce item, arranged in accordance with at least some embodiments described in the present disclosure. The computing system 900 may represent an example configuration of the computing system 104 described above. In a very basic configuration 902, the computing system 900 may include one or more processors 904 and a system memory 906. A memory bus 908 may be used for communicating between the processor 904 and the system memory 906.

Depending on the desired configuration, the processor 904 may be of any type including, but not limited to, a microprocessor (μP), a microcontroller (μC), a digital signal processor (DSP), or any combination thereof. The processor 904 may include one more levels of caching, such as a level one cache 910 and a level two cache 912, a processor core 914, and registers 916. An example processor core 914 may include an arithmetic logic unit (ALU), a floating point unit (FPU), a digital signal processing core (DSP Core), or any combination thereof. An example memory controller 918 may also be used with the processor 904, or in some implementations the memory controller 918 may be an internal part of the processor 904.

Depending on the target configuration, the system memory 906 may be of any type including, but not limited to, volatile memory (such as RAM), non-volatile memory (such as ROM, flash memory, etc.), or any other type of non-transitory computer-readable medium and any combination thereof. The system memory 906 may include an operating system 920, one or more applications 922, and program data 924. The application 922 may include a determination application 926 that may include instructions (executable by a processor such as the processor 904) pertaining to determining a ripeness of a produce item. The program data 924 may include ripeness data 928 that may be useful for determining ripeness of a produce item, as is described in the present disclosure. For example, the ripeness data may include a look-up table such as described above. In some embodiments, the application 922 may be arranged to operate with the program data 924 on the operating system 920 such that the pressure adjustment and other printer-related operations may be performed. This described basic configuration 902 is illustrated in FIG. 9 by those components within the inner dashed line.

The computing system 900 may have additional features or functionality, and additional interfaces to facilitate communications between the basic configuration 902 and any required devices and interfaces. For example, a bus/interface controller 930 may be used to facilitate communications between the basic configuration 902 and one or more data storage devices 932 via a storage interface bus 934. Data storage devices 932 may be removable storage devices 936, non-removable storage devices 938, or a combination thereof. Examples of removable storage and non-removable storage devices include magnetic disk devices such as flexible disk drives and hard-disk drives (HDDs), optical disk drives such as compact disk (CD) drives or digital versatile disk (DVDs) drives, solid state drives (SSDs), and tape drives to name a few. Example computer storage media may include volatile and nonvolatile, removable and non-removable media implemented in any method or technology for storage of information, such as computer-readable instructions, data structures, program modules, or other data.

The system memory 906, the removable storage devices 936, and the non-removable storage devices 938 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by the computing system 900. Any such computer storage media may be part of the computing system 900.

The computing system 900 may also include an interface bus 940 for facilitating communication from various interface devices (e.g., output devices 942, peripheral interfaces 944, and communication devices 946) to the basic configuration 902 via the bus/interface controller 930. Example output devices 942 include a graphics processing unit 948 and an audio processing unit 950, which may be configured to communicate to various external devices such as a display or speakers via one or more A/V ports 952. Example peripheral interfaces 944 include a serial interface controller 954 or a parallel interface controller 956, which may be configured to communicate with external devices such as input devices (e.g., keyboard, mouse, pen, voice input device, touch input device, etc.) or other peripheral devices (e.g., printer, scanner, etc.) via one or more I/O ports 958. An example communication device 946 includes a network controller 960, which may be arranged to facilitate communications with one or more other computing systems 962 over a network communication link via one or more communication ports 964.

The network communication link may be one example of a communication media. Communication media may typically be embodied by computer-readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term “computer-readable media,” as used herein, may include both storage media and communication media.

The computing system 900 may be implemented as a portion of a small-form factor portable (or mobile) electronic device such as a cell phone, a personal data assistant (PDA), a personal media player device, a wireless web-watch device, a personal headset device, an application-specific device, or a hybrid device that includes any of the above functions. The computing system 900 may also be implemented as a personal computer including both laptop computer and non-laptop computer configurations.

The present disclosure is not to be limited in terms of the particular embodiments described in the present disclosure, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure includes the terms of the appended claims, along with the full scope of equivalents to which such claims are entitled. The present disclosure is not limited to particular methods, reagents, compounds, compositions, or biological systems, which can, of course, vary. The terminology used in the present disclosure is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In general, terms used in the present disclosure, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Further, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Additionally, virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, the disclosure is also thereby described in terms of any individual member or subgroup of members of the Markush group.

For any and all purposes, such as in terms of providing a written description, all ranges disclosed herein also encompass any and all possible sub ranges and combinations of sub ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. Also all language such as “up to,” “at least,” and the like may include the number recited and refer to ranges which can be subsequently broken down into sub ranges as discussed above. Finally, a range may include each individual member. Thus, for example, a group having 1-3 cells may refer to groups having 1, 2, or 3 cells. Similarly, a group having 1-5 cells refers to groups having 1, 2, 3, 4, or 5 cells, and so forth.

From the foregoing, various embodiments of the present disclosure have been described herein for purposes of illustration, and various modifications may be made without departing from the scope and spirit of the present disclosure. Accordingly, the various embodiments disclosed herein are not intended to be limiting, with the true scope and spirit being indicated by the following claims.

What is claimed is:

1. A measurement circuit, comprising:
an input circuit configured to obtain an electrical signal as an input signal;
a sensing circuit electrically coupled to the input circuit and configured to:
generate a reference signal based on the input signal; and
generate a device-under-test signal based on the input signal and based on a device-under-test capacitance between a first contact point of the sensing circuit and a second contact point of the sensing circuit, wherein the first contact point is electrically coupled to a first contact mechanism configured to contact a first contact area of a device-under-test and wherein the second contact point is configured to be electrically coupled to a second contact mechanism configured to contact a second contact area of the device-under-test;
a comparison circuit electrically coupled to the sensing circuit and configured to generate a comparison signal based on a comparison between the reference signal and the device-under-test signal; and
an output circuit electrically coupled to the comparison circuit and configured to emit an output signal that is based on the comparison signal.

2. The measurement circuit of claim 1, wherein:
the device-under-test includes a produce item;
the device-under-test capacitance is based on a ripeness of the produce item; and
the output signal indicates the ripeness of the produce item.

3. The measurement circuit of claim 1, wherein:
the comparison signal includes an alternating current (AC) signal; and
the output circuit is configured to convert the comparison signal from AC to direct current (DC) to generate the output signal.

4. The measurement circuit of claim 1, wherein the input signal includes an alternating current (AC) signal with a peak-to-peak voltage that is based on the device-under-test.

5. The measurement circuit of claim 1, wherein the input signal includes an alternating current (AC) signal with a frequency that is based on the device-under-test.

6. The measurement circuit of claim 1, wherein the sensing circuit includes:
a resistive element electrically coupled to a first node to which an output of the input circuit is coupled, wherein a resistance of the resistive element is based on the device-under-test;
a capacitive element electrically coupled between the resistive element and a ground of the measurement circuit, wherein a capacitance of the capacitive element is based on the device-under-test; and
a reference node between the resistive element and the capacitive element, wherein the reference signal is output by the sensing circuit at the reference node.

7. The measurement circuit of claim 1, wherein the sensing circuit includes:
the first contact point electrically coupled to a device-under-test node, wherein the device-under-test signal is output by the sensing circuit at the device-under-test node;
a resistive element electrically between the device-under-test node and a first node to which an output of the input circuit is coupled, wherein a resistance of the resistive element is based on the device-under-test; and
the second contact point electrically coupled to a ground of the measurement circuit.

8. A measurement system comprising:
a contact system including:
a first contact mechanism configured to contact a produce item at a first contact area of a surface of the produce item, wherein the first contact mechanism is electrically conductive and is configured such that a size of the first contact area in contact with the first contact mechanism changes according to a ripeness of the produce item;
a second contact mechanism configured to contact the produce item at a second contact area of the surface of the produce item, wherein the second contact mechanism is electrically conductive; and
a measurement circuit including a first contact point electrically coupled to the first contact mechanism and including a second contact point electrically coupled to the second contact mechanism, wherein the measurement circuit is configured to generate an output signal based on an electrical property between the first contact point and the second contact point.

9. The measurement system of claim 8, further comprising a computing system communicatively coupled to the measurement circuit and configured to determine the ripeness of the produce item based on the output signal of the measurement circuit.

10. The measurement system of claim 8, wherein the first contact mechanism includes:
an electrically conductive ball; and
a deployment mechanism configured to press the electrically conductive ball against the surface of the produce item.

11. The measurement system of claim 10, further comprising an insulative guard ring disposed around the electrically conductive ball, wherein the insulative guard ring is configured to contact the surface of the produce item.

12. The measurement system of claim 8, wherein the first contact mechanism includes an electrically conductive roller configured to support at least a portion of the produce item such that a weight of the produce item presses the surface of the produce item against the electrically conductive roller.

13. The measurement system of claim 8, wherein the first contact mechanism includes an electrically conductive roller and a plurality of insulative rings disposed around the electrically conductive roller.

14. The measurement system of claim 8, wherein the measurement circuit further includes:
an input circuit configured to obtain an electrical signal as an input signal;
a sensing circuit electrically coupled to the input circuit and configured to:
output a reference signal based on the input signal; and
output a device-under-test signal based on the input signal and based on a device-under-test capacitance between the first contact point and the second contact point;
a comparison circuit electrically coupled to the sensing circuit and configured to generate a comparison signal based on a comparison between the reference signal and the device-under-test signal; and
an output circuit electrically coupled to the comparison circuit and configured to generate the output signal based on the comparison signal.

15. The measurement system of claim 14, wherein the input signal includes an alternating current (AC) signal with a peak-to-peak voltage that is based on the produce item.

16. The measurement system of claim 14, wherein the input signal includes an alternating current (AC) signal with a frequency that is based on the produce item.

17. The measurement system of claim 14, wherein the sensing circuit includes:
a resistive element electrically coupled to a first node to which an output of the input circuit is coupled, wherein a resistance of the resistive element is based on the produce item;
a capacitive element electrically coupled between the resistive element and a ground of the measurement circuit, wherein a capacitance of the capacitive element is based on the produce item; and
a reference node between the resistive element and the capacitive element, wherein the reference signal is output by the sensing circuit at the reference node.

18. The measurement system of claim 14, wherein the sensing circuit includes:
the first contact point electrically coupled to a device-under-test node, wherein the device-under-test signal is output by the sensing circuit at the device-under-test node;
a resistive element electrically between the device-under-test node and a first node to which an output of the input circuit is coupled, wherein a resistance of the resistive element is based on the produce item; and
the second contact point electrically coupled to a ground of the measurement circuit.

19. A method comprising:
obtaining an electrical signal as an input signal, wherein the input signal includes an alternating current (AC) signal with one or more of the following based on a device-under-test: a peak-to-peak voltage and a frequency;
generating a reference signal based on the input signal; and
generating a device-under-test signal based on the input signal and based on a device-under-test capacitance between a first contact point and a second contact point, wherein the first contact point is electrically coupled to a first contact mechanism configured to contact a first contact area of the device-under-test and wherein the second contact point is configured to be electrically coupled to a second contact mechanism configured to contact a second contact area of the device-under-test;
generating a comparison signal based on a comparison between the reference signal and the device-under-test signal; and
emitting an output signal that is based on the comparison signal.

20. The method of claim 19, wherein the device-under-test includes a produce item and the method further comprises determining a ripeness of the produce item based on the output signal.
